(19) United States
(12) Patent Application Publication
(10) Pub. No.: US 2014/0354788 A1
(43) Pub. Date: Dec. 4, 2014

(54) ENDOSCOPE SYSTEM

(71) Applicant: FUJIFILM CORPORATION, Tokyo (JP)

(72) Inventor: Takashi YANO, Ashigarakami-gun (JP)

(73) Assignee: FUJIFILM CORPORATION, Tokyo (JP)

(21) Appl. No.: 14/294,776

(22) Filed: Jun. 3, 2014

(30) Foreign Application Priority Data
Jun. 4, 2013 (JP) 2013-117892

(51) Int. Cl. A61B 1/06; H04N 5/374; A61B 1/04

(52) U.S. Cl. CPC A61B 1/0661; A61B 1/043; H04N 5/374
USPC 348/68

(57) ABSTRACT

An endoscope unit includes a sequence pattern setting section in which a combination of sequence pattern which includes a plurality of parameters, each corresponding to imaging conditions of the image sensor on a frame-by-frame basis according to the type of the light source and control signal is set, an imaging condition setting section in which imaging conditions corresponding to each parameter are set, and an imaging control section that obtains the sequence pattern based on a control signal outputted from a control section of a processor unit, reads out the imaging conditions corresponding to each parameter included in the sequence pattern from the imaging condition setting section, and controls the imaging operation of the image sensor on a frame-by-frame basis based on the imaging conditions.

### CROSS-REFERENCE TO RELATED APPLICATIONS

The present application claims priority under 35 U.S.C. §119 to Japanese Patent Application No. 2013-117892 filed on Jun. 4, 2013. Each of the above application(s) is hereby expressly incorporated by reference, in its entirety, into the present application.

### BACKGROUND OF THE INVENTION

1. Field of the Invention

The present invention relates to an endoscope system in which light of different wavelengths is projected onto an observation target area by sequentially switching a plurality of types of light sources and image signals due to the projection of each light are obtained on a frame-by-frame basis.

2. Description of the Related Art

Endoscope systems for observing tissues in the bodies have been widely known and electronic endoscope systems that obtain a visual image by imaging an observation target area by the use of an image sensor and display the visual image on a monitor screen are widely put into practical use.

Here, a narrow band imaging (NBI) system is drawing attention as one of the endoscope systems described above. The system includes narrow band-pass filters, projects two types of narrow band light, blue and green light, through the narrow band-pass filters, and forms a spectral image by performing predetermined processing on the image signals obtained by the projection of the narrow band light. According to such spectral images, fine structures and the like which have not been obtained in the past may be observed in digestive organs such as stomachs, large intestines, and the like.

Further, another type of endoscope system is proposed in which ICG (indocyanine green) is injected into an observation target area in advance and an ICG fluorescence image is obtained by projecting excitation light of near infrared light onto the observation target area in order to observe blood vessel runs and blood flows under fat, lymph vessels, lymph flows, bile duct runs, bile flows, and the like that do not appear on ordinary images. Still another type of endoscope system is also proposed in which a fluorescence image is obtained by projecting excitation light onto an observation target area and detecting autofluorescence emitted from the observation target area.

In the endoscope systems that project special light, such as narrow band light or excitation light described above, for example, International Patent Publication No. 11/072473 proposes an endoscope system in which an ordinary image and a special image are alternately captured by alternately switching and projecting white light and special light to an observation target area on a frame-by-frame basis in order to capture and display both an ordinary image through projection of white light and a special image through projection of special light.

In view of the fact that the light intensity of reflection light of the narrow band light or the fluorescence is weak in comparison with the light intensity of reflection light of the white light and the brightness of the special image becomes dark, International Patent Publication No. 11/072473 proposes a method for increasing the brightness of the special image by changing imaging conditions, such as extending the exposure time of the image sensor longer for capturing the special image than for capturing the ordinary image, reading the image signal of the same line a plurality of times and adding them together when capturing the special image, or the like.

### SUMMARY OF THE INVENTION

In the endoscope system described in International Patent Publication No. 11/072473, however, a control signal is outputted from the processor unit to the image sensor on a frame-by-frame basis for switching the imaging conditions when switching the imaging conditions of the image sensor according to the switching of white light and special light on a frame-by-frame basis. In the case where a control signal is outputted from the processor unit to the image sensor on a frame-by-frame basis, as described above, it is necessary that the control signal for switching the imaging conditions needs to be received by the image sensor before the start of the imaging of next frame. If the receive timing of the control signal by the image sensor is delayed due to, for example, the occurrence of certain interrupt processing in a control section of the processor unit, the imaging condition is changed in the middle of the imaging operation of the next frame and, in this case, the image will be collapsed.

The output of control signal from the processor unit on a frame-by-frame basis, as described above, causes the burden on the control section of the processor unit to be increased.

The present invention has been developed in view of the circumstances described above, and it is an object of the present invention to provide, in endoscope systems in which a plurality of types of light sources is sequentially switched to project the light on an observation target area and imaging conditions of the image sensor are switched according to the projection of each light, an endoscope system capable of reducing the control burden on the processor unit without incurring image collapse described above.

An endoscope system of the present invention includes a light source unit that emits light by sequentially switching a plurality of types of light sources, an endoscope unit equipped with an image sensor, and a processor unit connected to the endoscope unit as an external unit and includes a control section that controls the endoscope unit, wherein the endoscope unit includes:

a sequence pattern setting section in which a combination of sequence pattern and predetermined control signal to be outputted from the control section is set, the sequence pattern including a plurality of parameters, each corresponding to imaging conditions of the image sensor on a frame-by-frame or a plurality of frames-by-frames basis according to the type of light source;

an imaging condition setting section in which a plurality of types of parameters and imaging conditions corresponding to each parameter are set in association with each other; and

an imaging control section that obtains the sequence pattern based on the control signal outputted from the control section of the processor unit, sequentially reads out the imaging conditions corresponding to each parameter included in the obtained sequence pattern from the imaging condition setting section, and controls the imaging operation of the image sensor on a frame-by-frame or a plurality of frames-by-frames basis based on the sequentially read out imaging conditions.

In the endoscope system of the present invention described above, the sequence pattern setting section may be a section in which a plurality of types of combinations of sequence pattern and control signal is set, and the imaging control section may select and obtain one sequence pattern from the plurality of types of sequence patterns.

Further, the endoscope unit may include a register in which the imaging conditions sequentially read out from the imaging condition setting section on a frame-by-frame or a plurality of frames-by-frames basis are temporarily stored and sequentially updated.

Still further, the imaging control section may output information of the currently set imaging conditions to the processor unit.

Further, the imaging control section may output the information of the imaging conditions by superimposing the information on an image signal outputted from the image sensor.

Still further, the imaging control section may output the information of the imaging conditions during a blanking time of the image sensor.

Further, the processor unit may include an imaging condition judgment section that judges whether or not the information of the imaging conditions outputted from the imaging control section is correct.

Still further, the endoscope unit may include an amplifier that amplifies an image signal outputted from the image sensor, and one of the imaging conditions may be gain of the amplifier.

Further, the imaging conditions may include at least one of exposure time of the image sensor and reading target pixel information of the image sensor.

Still further, the reading target pixel information may be information of reading a plurality of pixel signals by adding the signals together or information of reading a plurality of pixel signals by averaging the signals.

Further, at least one of the sequence pattern setting section, the imaging condition setting section, and the imaging control section may be formed on one IC (Integrated Circuit) chip with the image sensor.

Still further, a CMOS (Complementary Metal-Oxide Semiconductor) may be used as the image sensor.

Further, the light source unit may include at least two of a white light source, a narrow band light source that emits narrow band light, and an excitation light source that emits excitation light for causing an observation target area to generate fluorescence.

Still further, the light source unit may include a red light source that emits red light, a green light source that emits green light, and a blue light source that emits blue light, as the plurality of types of light sources.

According to the endoscope system of the present invention, a combination of sequence pattern which includes a plurality of parameters, each corresponding to imaging conditions of the image sensor on a frame-by-frame or a plurality of frames-by-frames basis according to the type of light source and predetermined control signal to be outputted from the control section is preset in the sequence pattern setting section of the endoscope unit, a plurality of types of parameters and imaging conditions correspond to each parameter are set in the imaging condition setting section of the endoscope unit in association with each other, and the imaging control section of the endoscope unit obtains the sequence pattern based on the control signal outputted from the control section of the processor unit, sequentially reads out the imaging conditions corresponding to each parameter included in the obtained sequence pattern from the imaging condition setting section, and controls the imaging operation of the image sensor on a frame-by-frame or a plurality of frames-by-frames basis based on the sequentially read out imaging conditions. This requires the control signal output from the processor unit to the endoscope unit only once in the beginning, and eliminates the need to perform communication of control signal on a frame-by-frame basis as in the past.

Consequently, an image collapse due to a change in the imaging conditions in the middle of imaging one frame arising from a receiving error of control signal may be prevented and the burden on the control section of the processor unit may be reduced at the same time.

### BRIEF DESCRIPTION OF THE DRAWINGS

FIG. 1 is an external view of an embodiment of the endoscope system of the present invention, illustrating the schematic configuration thereof.

FIG. 2 is a cross-sectional view of a flexible tube portion of an insertion section, illustrating the inside thereof.

FIG. 3 illustrates the configuration of a distal end portion of the insertion section.

FIG. 4 is a longitudinal sectional view of the distal end portion of the insertion section, illustrating the inside thereof.

FIG. 5 illustrates a specific configuration of an image sensor.

FIG. 6 illustrates, by way of example, combinations of control signal and sequence pattern set in a sequence pattern setting section.

FIG. 7 illustrates, by way of example, imaging conditions set in association with each parameter.

FIG. 8 illustrates, by way of example, imaging conditions temporarily stored in a register.

FIG. 9 is a block diagram of processor unit and light source unit of the endoscope system shown in FIG. 1, illustrating the internal configurations thereof.

FIG. 10 is a drawing for explaining the operation of an embodiment of the endoscope system of the present invention.

FIG. 11 illustrates, by way of example, color filters installed on the image sensor.

FIG. 12 is a drawing for explaining update delay of imaging conditions in the register.

FIG. 13 illustrates a modification of an embodiment of the endoscope system of the present invention.

FIG. 14 illustrates an example in which imaging conditions are superimposed during blanking times.

FIG. 15 illustrates a modification of an embodiment of the endoscope system of the present invention.

FIG. 16 illustrates a modification of an embodiment of the endoscope system of the present invention.

### DESCRIPTION OF THE PREFERRED EMBODIMENTS

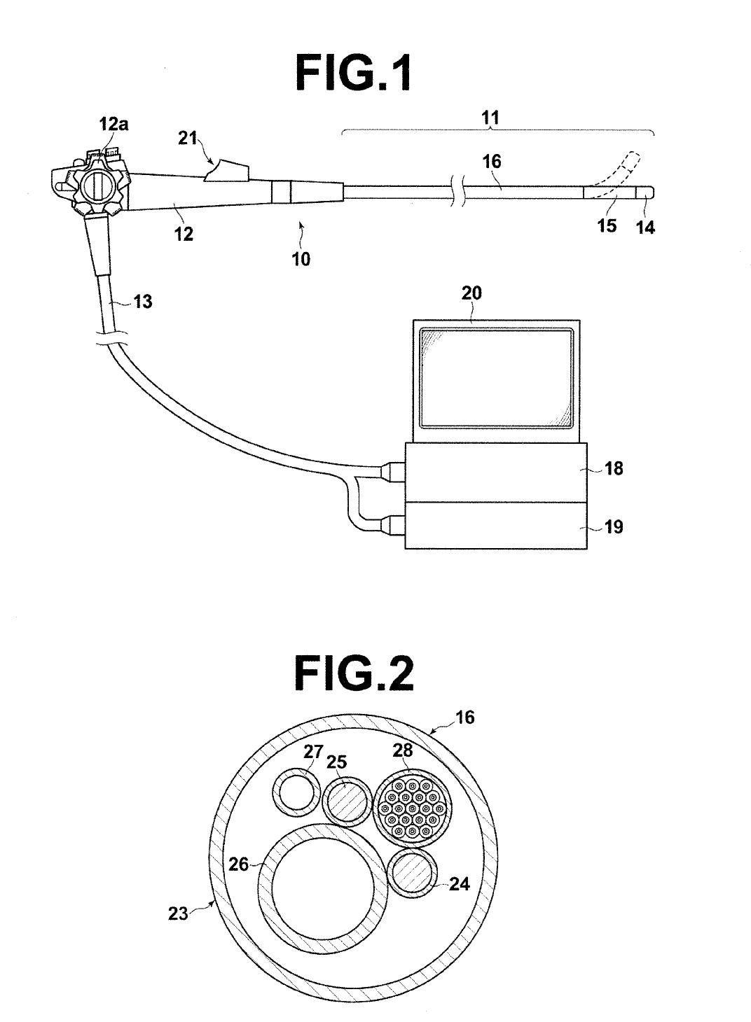

Hereinafter, an embodiment of the endoscope system of the present invention will be described in detail with reference to the accompanying drawings. The endoscope system of the present embodiment has a characteristic feature in the control method of imaging conditions for the image sensor according to the switching of a plurality of types of light sources. But the configuration of the entire system will be described first. FIG. 1 is an external view of the endoscope system of the present embodiment, illustrating the schematic configuration thereof.

As illustrated in FIG. 1, the endoscope system of the present embodiment includes an endoscope unit 10, a universal cable 13 whose one end is to be connected to the endoscope unit 10, a processor unit 18 and a light source unit 19 to which the other end of the universal cable 13 is to be connected, and a monitor 20 that displays an image based on image signals outputted from the processor unit 18.

The endoscope unit 10 includes an insertion section 11 and an operation section 12 that receives a given operation from the operator. The insertion section 11 is formed into a tubular shape, and more specifically, the insertion section 11 includes a distal rigid portion 14, a bend portion 15, and a flexible tube portion 16 from the distal end as shown in FIG. 1.

The distal rigid portion 14 is formed of a rigid metal material or the like, while the flexible tube portion 16 is a portion that connects the operation section 12 with the bend portion 15 in an elongated fashion with a small diameter and has flexibility. The bend portion 15 bends when an angle wire provided inside the insertion section 11 is pushed or pulled in association with the operation of an angle knob 12a provided on the operation section 12. This causes the distal rigid portion 14 to be directed to a desired direction within the body and a desired observation target area is imaged by an image sensor, to be described later, provided in the rigid tip portion 14. Further, the operation section 12 has a forceps opening 21 through which a treatment tool is to be inserted, and the forceps opening 21 is connected to a forceps tube 26, to be described later, disposed in the insertion section 11.

FIG. 2 is a cross-sectional view of the flexible tube portion of the insertion section, illustrating the inside thereof. As illustrated in FIG. 2, the flexible tube portion 16 includes a flexible tube 23 in which a plurality of contents, such as light guides 24, 25 for guiding illumination light to the illumination lens in the distal rigid portion 14, a forceps tube 26, a gas/liquid feed tube 27, a multi-core cable 28, and the like, are loosely inserted. The multi-core cable 28 is mainly a collection of control signal wiring for sending control signals from the processor unit 18 to drive the image sensor and image signal wiring for sending image signals captured by the image sensor to the processor unit 18, in which the plurality of signal wirings are covered by a protective coating.

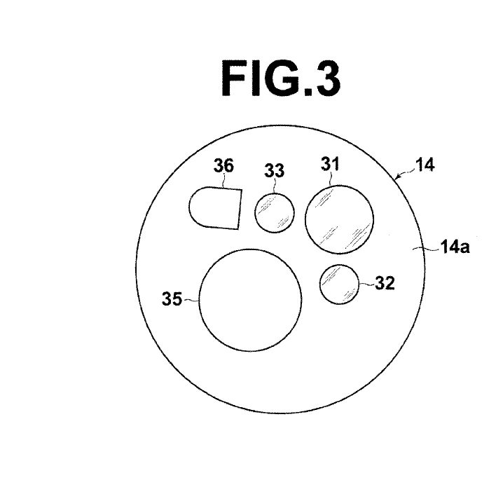

FIG. 3 illustrates a distal end face 14a of the distal rigid portion 14. As illustrated in FIG. 3, the distal end face 14a of the distal rigid portion 14 includes an observation window 31, illumination windows 32, 33, a forceps exit 35, a gas/liquid feed nozzle 36, and the like. The observation window 31 includes a part of an objective optical system for introducing image light of an observation target area in the body. The illumination windows 32, 33 include apart of illumination lens and project illumination light emitted from the light source unit 19 and guided by the light guides 24, 25 onto an observation target area in the body. The forceps output 35 is communicated with the forceps opening 21 provided in the operation section 12 via the forceps tube 26. The gas/liquid feed nozzle 36 sprays cleaning water or air for removing dirt from the observation window 31 by operating a gas/liquid feed button provided on the operation section 12. Note that a gas/liquid feed unit for feeding the liquid or gas to be sprayed from the gas/liquid feed nozzle 36 is omitted in the drawing.

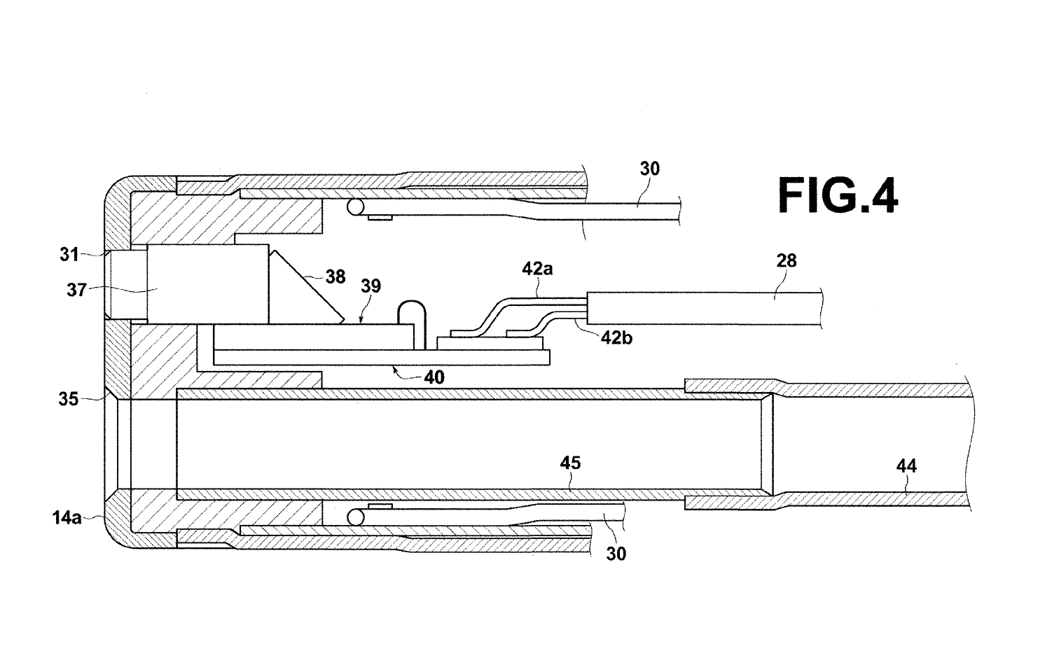

FIG. 4 is a longitudinal sectional view of the distal end portion of the insertion section, illustrating the inside thereof. As illustrate in FIG. 4, an objective optical system 37 is disposed at a position opposite to the observation window 31. Illumination light projected from the illumination window 32, 33 is reflected at the observation target area and enters into the observation window 31. The image of the observation target area entered from the observation window 31 is incident on a prism 38 through the objective optical system 37 and formed on the image plane of the image sensor 39 by bending inside the prism 38.

The circuit board 40 is a board on which a wiring pattern is formed for relaying control signals to be inputted to the image sensor 39 and image signals to be outputted from image sensor 39 from/to the control signal wiring and image signal wiring of the multi-core cable 28.

A control signal wiring 42a and an image signal wiring 42b are exposed from an end of the multi-core cable 28 disposed parallel to a longitudinal direction, and the control signal wiring 42a and the image signal wiring 42b are electrically connected to the wiring pattern of the circuit board 40.

A flexible tube of synthetic resin 44 is disposed inside the bend portion 15. One end of the flexible tube 44 is connected to the forceps tube 26 and the other end is connected to a rigid tube 45 disposed inside the distal rigid portion 14. The rigid tube 45 is fixedly disposed inside the distal rigid portion 14 and the tip is connected to the forceps exit 35.

The image sensor 39 of the present embodiment will now be described in detail.

The image sensor 39 performs a photoelectric conversion on an image formed on the image plane and outputs image signals with respect to each frame according to a given synchronization signal outputted from a control section 56 of the processor unit 18. Color filters of three primary colors, red (R), green (G), and blue (B), are arranged in Bayer pattern or honeycomb pattern on the image plane of the image sensor 39.

As for the image sensor 39, a CMOS (Complementary Metal Oxide Semiconductor) sensor, a CCD sensor, or the like may be used. In the present embodiment, a CMOS sensor having Bayer-arranged color filters will be used as the image sensor 39.

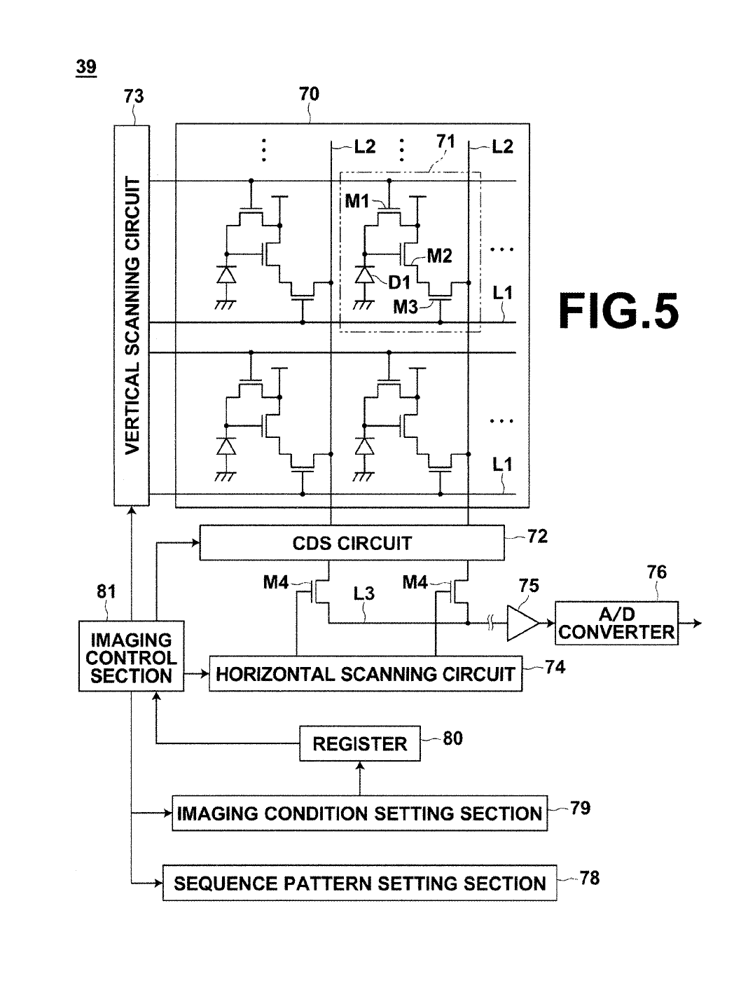

FIG. 5 illustrates a specific configuration of the image sensor 39 of the present embodiment. As illustrated in FIG. 5, the image sensor 39 of the present embodiment includes a pixel section 70 in which pixel circuits 71 are disposed in matrix, a CDS circuit 72 that performs correlated double sampling on image signals outputted from each pixel circuit 71, a vertical scanning circuit 73 that controls scanning of the pixel section 70 in a vertical direction and reset operation of the pixel section 70, and a horizontal scanning circuit 74 that controls scanning of the pixel section 70 in a horizontal direction. The image sensor 39 further includes an amplifier 75 that amplifies and outputs the pixel signal outputted from the CDS circuit 72, an A/D converter that converts the pixel signal in analog form outputted from the amplifier 75 to a digital signal and outputs the digitized pixel data, and an imaging control section 81 that controls the imaging operation of the entire image sensor 39.

The pixel circuit 71 includes a photodiode D1, a reset transistor M1, a drive transistor M2, and a line selection transistor M3. The line selection transistor M3 of each pixel circuit 71 is connected to a scanning line L1 and drive transistor M2 is connected to a signal line L2, and each pixel circuit 71 is sequentially scanned by the vertical scanning circuit 73 and the horizontal scanning circuit 74.

The imaging control section 81 generates and outputs control signals to be inputted to the vertical scanning circuit 73 and the horizontal scanning circuit 74 for scanning the rows and columns of the pixel circuits 71, a control signal to be inputted to the vertical scanning circuit 73 for resetting signal charges accumulated in the photodiodes D1, a control signal to be inputted to the CDS circuit 72 for controlling connection between the pixel circuits 71 and the CDS circuit 72, and the like.

The CDS circuit 72 is dividedly provided with respect to each signal line L2, and performs correlated double sampling on the pixel signals outputted from each pixel circuit 71 connected to the scanning line L1 selected by the vertical scanning circuit 73 and sequentially outputs the pixel signals subjected to the correlated double sampling to the amplifier 75 according to the horizontal scanning signal outputted from the horizontal scanning circuit 74. The horizontal scanning circuit 74 performs ON/OFF control of column select transistors M4 provided between the CDS circuit 72 and an output bus line L3 connected to the amplifier 75 by the horizontal scanning signal. All rows are scanned by the horizontal scanning circuit 73 and the pixel signals of each row are sequentially horizontal-scanned by the horizontal scanning circuit 74, whereby image signals of one frame are outputted.

As described above, the amplifier 75 amplifies and outputs the pixel signals outputted from the CDS circuit 72 which is a variable gain amplifier constructed variable in gain at the time of amplifying the pixel signals. The gain of the amplifier 75 is set by a control signal from the imaging control section 81. The pixel signal outputted from the amplifier 75 is converted to a digital signal by the A/D converter 76 and outputted to the processor unit 18 via the image signal wiring.

The image sensor 39 further includes a sequence pattern setting section 78, an imaging condition setting section 79, and a register 80.

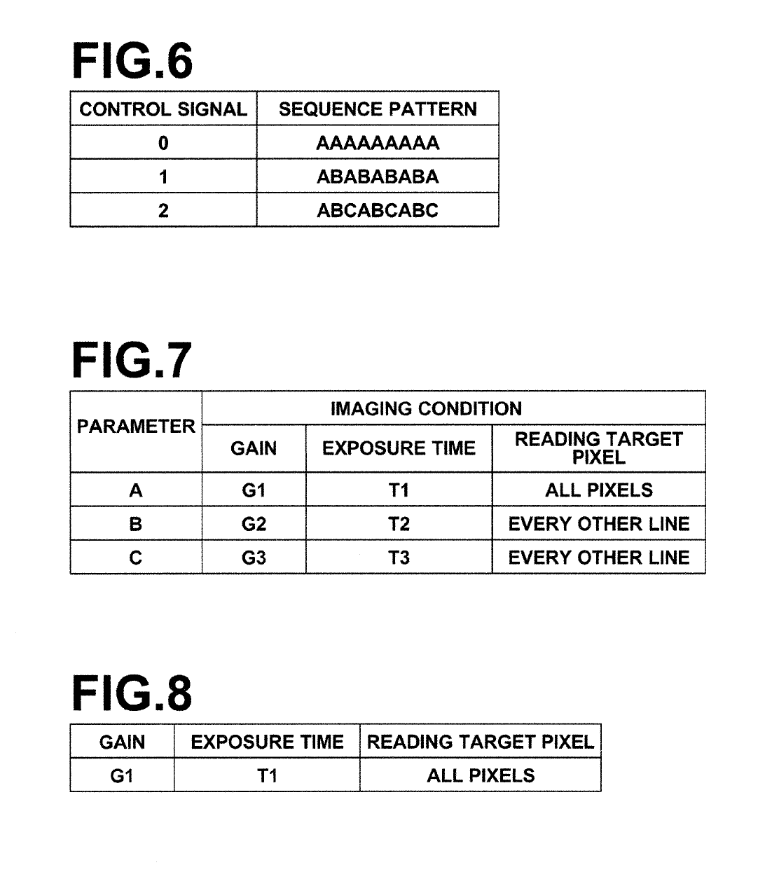

As illustrated in FIG. 6, the sequence pattern setting section 78 is a section in which a plurality of types of combinations of predetermined control signal outputted from a control section 56, to be described later, of the processor unit 18 and sequence pattern is set.

As shown in FIG. 6, the sequence pattern is a pattern in which a plurality of parameters, such as A, B, C, and the like, is arranged. Each of these parameters is set in association with the imaging condition which corresponds to the type of light source in the light source unit 19. The imaging conditions of the image sensor 39 corresponding to parameter “A”, the imaging conditions of the image sensor 39 corresponding to parameter “B”, and the imaging conditions of the image sensor 39 corresponding to parameter “C” differ from each other. The imaging conditions corresponding to each parameter are applied to the imaging operation on a frame-by-frame basis or a plurality of frames-by-frames basis. In the present embodiment, the imaging conditions corresponding to each parameter are applied to imaging operation on a frame-by-frame basis, but the imaging conditions corresponding to each parameter may be applied on a plurality of frames-by-frames basis.

That is, as the sequence pattern includes a plurality of parameters disposed therein, as described above, the sequence pattern refers to imaging conditions encoded by the parameters, such as A, B, C, and the like, to be applied to the imaging operation of each frame sequentially performed in time series. The number of parameters in the sequence pattern may be set and changed arbitrarily by the user through an input section 55 of the processor unit 18

The control signal corresponding to each sequence pattern is outputted from the control section 56 of the processor unit 18, as described above, and is represented by a single digit number, such as 0, 1, and 2 as shown in FIG. 6. Note that the control signal is not necessarily a single digit number and any signal may be basically used if it is a simple control signal which can be transmitted instantaneously.

FIG. 6 shows three types of combinations of control signal and sequence pattern, but two types of combinations or four or more types of combinations may be set. The combination of control signal and sequence pattern may be set and changed arbitrarily by the user through the input section 55 of the processor unit 18.

As illustrated in FIG. 7, the imaging condition setting section 79 is a section in which a plurality of parameters, such as A, B, C and the like and imaging conditions corresponding to each parameter are set in association with each other. The imaging conditions associated with each parameter are set according to the type of light source in the light source unit 19 and a plurality of imaging conditions, not limited to one, may be set, such as gain of the amplifier 75, exposure time of the pixel section 70 (shutter speed), reading target pixel information in the pixel section 70, as illustrated in FIG. 7. The imaging conditions are not limited to the three types shown in FIG. 7 and other imaging conditions may be set and the setting change may be made by the user through the input section 55 of the processor unit 18. Note that all of the imaging conditions corresponding to each parameter are not necessarily different from parameter to parameter and some of the imaging conditions may be common to different parameters. Specific contents of the imaging conditions corresponding to each parameter will be described in detail later.

The imaging control section 81 receives the aforementioned control signal (0, 1, 2, or the like) outputted from the control section 56 of the processor unit 18 via the control signal wiring 42a and selects one sequence pattern from a plurality of types of sequence patterns set in the sequence pattern setting section 78 based on the received control signal. For example, the imaging control section 81 selects sequence pattern “AAAAAAAAA” if control signal “0” is received, selects sequence pattern “ABABABABA” if control signal “1” is received, and selects sequence pattern “ABCABCABC” if control signal “2” is received.

Then, the imaging control section 81 sequentially reads out the imaging conditions corresponding to each parameter included in the selected sequence pattern from the imaging condition setting section 79 and controls the imaging operation of the image sensor 39 on a frame-by-frame basis based on the sequentially read out imaging conditions. Note that the imaging conditions corresponding to each parameter read out from the imaging condition setting section 79 for each frame are temporarily stored sequentially in the register 80 on a frame-by-frame basis. FIG. 8 shows the state in which the imaging conditions corresponding to the parameter “A” are temporarily stored in the register 80.

The imaging control section 81 controls gain of the amplifier 75, exposure time of the pixel section 70, and reading target pixel in the pixel section 70 based on the imaging conditions temporarily stored in the register 80. Imaging conditions for each frame are temporarily stored and sequentially updated in the register 80. Note that, for the gain and exposure time, the values thereof are temporarily stored in the register while, for the reading target pixel information, a numerical value representing each reading target pixel information, such as 0, 1, 2, or the like, is stored in the register 80 and the imaging control section 81 controls the reading target pixel based on the numerical value. The imaging control section 81 includes a table or the like in which the aforementioned numerical values and read control signals are associated.

For control of the exposure time, for example, the time between the reset and read operations of each pixel circuit 71 of pixel section 70 may be controlled or, if the image sensor 39 is provided with a so-called electronic shutter function, the shutter speed of the electronic shutter may be controlled.

The image sensor 39 of the present embodiment is made of one IC chip on which each of all the sections shown in FIG. 5 is integrated. Note that, however, the image sensor 39 does not necessarily take a one-chip configuration and, for example, the imaging control section 81, the register 80, the imaging condition setting section 79, and the sequence pattern setting section 78 may be formed as another IC chip.

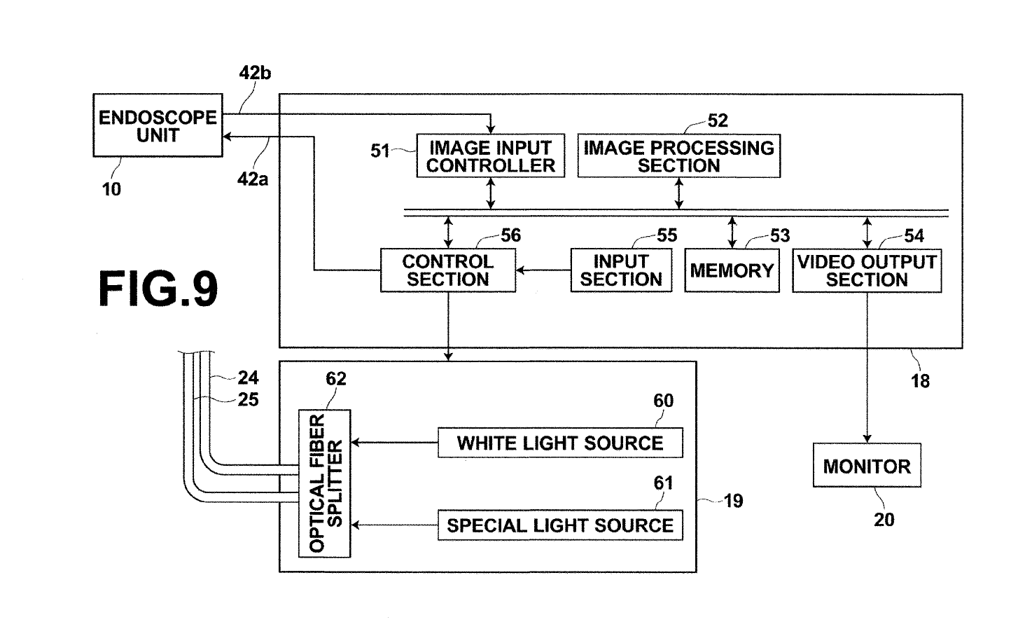

FIG. 9 schematically illustrates the internal configurations of the processor unit 18 and the light source unit 19. As illustrated in FIG. 9, the processor unit 18 includes an image input controller 51, an image processing section 52, a memory 53, a video output section 54, an input section 55, and a control section 56.

The image input controller 51 includes a line buffer with a given capacity and temporarily stores image signals of one frame outputted from the image sensor 39 of the endoscope unit 10. The image signals stored in the image input controller 51 are then stored in the memory 53 via the bus.

The image processing section 52 receives image signals of one frame read out from the memory 53 to perform predetermined image processing on the image signals and outputs the resultant image signals to the bus.

The video output section 54 receives the image signals outputted from the image processing section 52 via the bus to generate display control signals by performing predetermined processing on the received image signals and outputs the display control signals to the monitor 20.

The input section 55 receives user input, such as a predetermined operation instruction, a control parameter, and the like. The input section 55 of the present embodiment, in particular, receives input of the aforementioned combinations of control signal and sequence pattern, number of parameters in the sequence pattern, imaging conditions corresponding to each parameter, and the like.

The control section 56 controls the entire system and outputs, in particular, the aforementioned control signals (0, 1, 2, and the like) for controlling the imaging conditions of the image sensor 39 in the present embodiment, and further controls the switching of light emissions of a plurality of light sources according to the sequence pattern described above. The switching control of light emissions of a plurality of light sources in the light source unit 19 will be described later in detail.

As illustrated in FIG. 9, the light source unit 19 includes a white light source 60 that emits white light, a special light source 61 that emits special light, and an optical fiber splitter 62 that simultaneously inputs the received white light or special light to the light guides 24, 25 provided in the endoscope unit 10.

As for the white light source 60, for example, a halogen lamp may be used. The white light emitted from the halogen lamp has a wavelength range from 400 nm to 1800 nm. Note that other types of light sources, such as LED and the like, may be used other than the halogen lamp.

The special light source 61 emits light of a different wavelength range from that of the white light. The special light source 61 of the present embodiment is a narrow band light source that emits two types of narrow band light narrowed by narrow band-pass filters (hereinafter, simply referred to as the “narrow band light”). More specifically, the special light source 61 emits blue light narrowed to a wavelength range of about 400 nm to 430 nm and green light narrowed to a wavelength range of about 530 nm to 550 nm.

The light source unit 19 of the present embodiment performs switching between the white light from the white light source 60 and narrow band light from the special light source 61 based on a control signal outputted from the control section 56 of the processor unit 18. More specifically, white light from the white light source 60 is continuously emitted in an ordinary mode in which an ordinary image is captured by the projection of white light onto an observation target area. In a narrow band mode in which both a narrow band image, by the projection of narrow band light onto an observation target area, and an ordinary image are captured, white light from the white light source 60 and narrow band light from the special light source 61 are outputted alternately on a frame-by-frame basis.

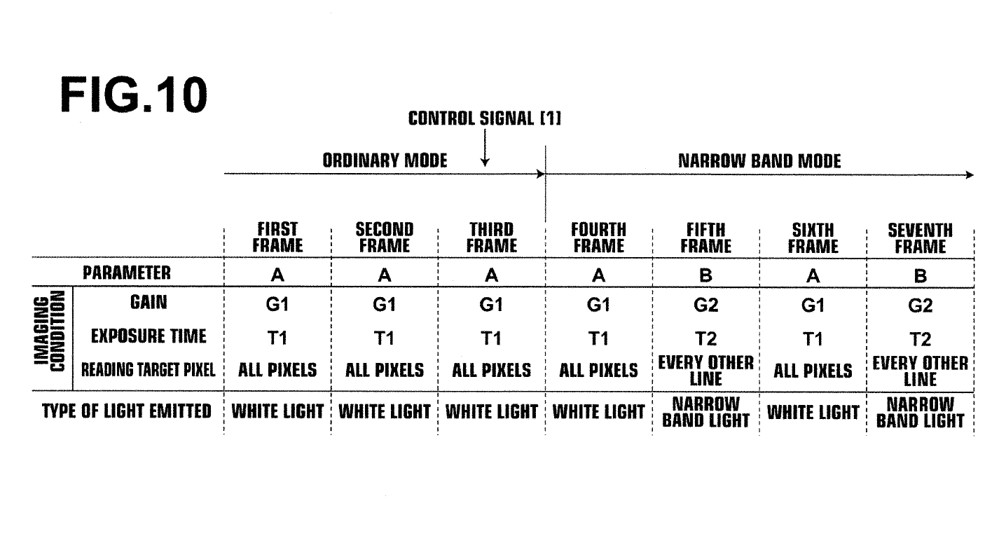

Next, the operation of the endoscope system of the present embodiment will be described with reference to FIG. 10. As the endoscope system of the present embodiment has a characteristic feature in the control method of imaging conditions for the image sensor 39 according to the switching of a plurality of types of light sources described above, the description will be made focusing on this point. More specifically, switching control from the ordinary mode in which an ordinary image is captured by, the projection of white light to the narrow band mode in which an ordinary image and a narrow band image are captured on a frame-by-frame basis by the alternate projection of white light and narrow band light.

Before describing the actual operation of the endoscope system, control signal outputted from the control section 56 of the processor unit when the endoscope system is switched to the ordinary mode or to the narrow band mode, sequence pattern corresponding to each mode, and imaging conditions corresponding to each parameter included in the sequence pattern will be described.

In the present embodiment, a control signal “0” is outputted from the control section 56 of the processor unit when the endoscope system is switched to the ordinary mode, and “AAAAAAAAA” shown in FIG. 6 is set as the sequence pattern corresponding to the ordinary mode. When the endoscope system is switched to the narrow band mode, a control signal “1” is outputted from the control section 56 of the processor unit and “ABABABABA” shown in FIG. 6 is set as the sequence pattern corresponding to the narrow band mode.

In the present embodiment, parameter “A” is set as the parameter corresponding to the imaging conditions when the white light is projected and parameter “B” is set as the parameter corresponding to the imaging conditions when the narrow band light is projected.

Then, as the imaging conditions corresponding to the parameter “A”, the gain of the amplifier 75 to G1, the exposure time of the pixel section 70 to T1, and information of all pixel reading are set as shown in FIG. 7. More specifically, it is assumed here that “1” is set as the gain G1 and “ 1/60 sec” is set as the exposure time T1. Further, as the imaging conditions corresponding to the parameter “B”, the gain of the amplifier 75 to G2, the exposure time of the pixel section 70 to T2, and information of every other line reading are set as shown in FIG. 7. More specifically, it is assumed here that “2” is set as the gain G2 and “ 1/30 sec” is set as the exposure time T2.

The reason why the gain of the amplifier 75 is set greater and the exposure time of the pixel section 70 is set longer for capturing a narrow band image than for capturing an ordinary image is that, as the narrow band light projected onto the observation target area in the narrow band mode is light passed through narrow band-pass filters as described above, the light intensity is weaker than that of the white light and the light intensity of the reflection light from the observation target area also becomes weak, so that a narrow band image of sufficient brightness may not be obtained.

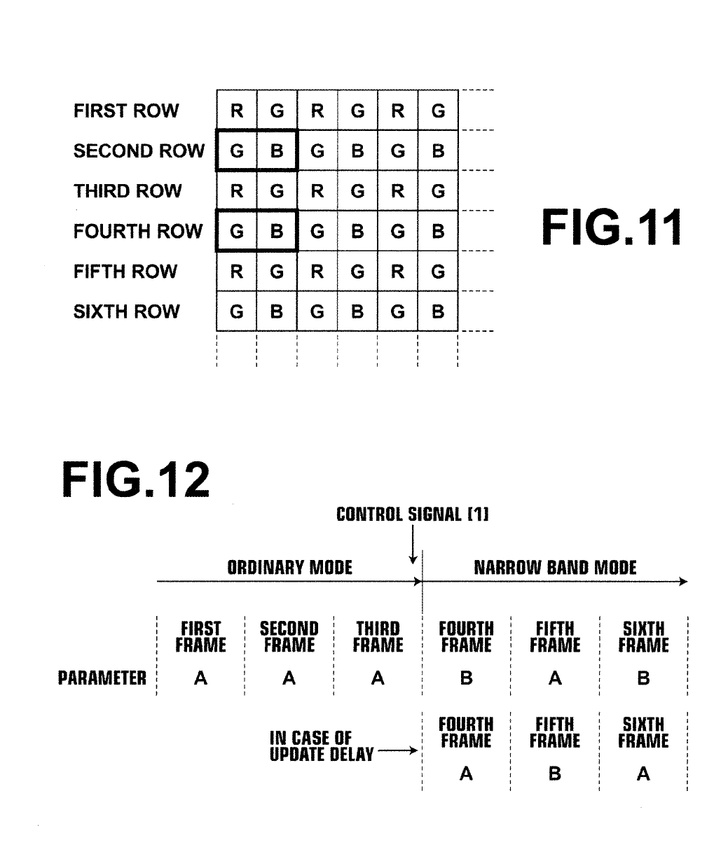

Further, as the exposure time T2 is set to a value twice that of the exposure time T1, the every other line reading is performed in the imaging operation of the narrow band image in the narrow band mode. In the case where the color filters installed on the image sensor 39 are arranged in Bayer pattern, as shown in FIG. 11, G (green) filters and B (blue) filters are disposed in even rows, only the even rows in FIG. 11 are set to be read out with the exposure time T2 which is twice the value of T1 in the imaging operation of the narrow band image.

Specific operation of the endoscope system will now be described.

If an instruction to perform normal mode imaging is set and inputted by the user from the input section 55 of the processor unit 18, the control section 56 outputs a control signal “0” to the imaging control section 81 of the image sensor 39.

If the control signal “0” is received, the imaging control section 81 selects the sequence pattern “AAAAAAAAA” from a plurality of types of sequence patterns set in the sequence pattern setting section 78.

Then, imaging conditions corresponding to the first parameter of the selected sequence pattern are read out by the imaging control section 81 from the imaging condition setting section 79. That is, gain G1, exposure time T1, and information of all pixel reading, which are imaging conditions corresponding to the parameter “A” shown in FIG. 7, are read out and temporarily stored in the register 80 by the imaging control section 81 as the imaging conditions of the first frame.

Then, white light is continuously emitted from the white light source 60 and the image sensor 39 is controlled based on the imaging conditions stored in the register 80 by the imaging control section 81, whereby imaging operation is performed for the first frame. More specifically, as illustrated in FIG. 10, the imaging control section 81 sets the gain of the amplifier 75 to G1, the exposure time of the pixel section 70 to T1, and performs an imaging operation through the all pixel reading for the first frame, whereby ordinary image signals are outputted.

Then, imaging conditions corresponding to the second parameter of the sequence pattern are read out by the imaging control section 81 from the imaging condition setting section 79. That is, as in the first frame, gain G1, exposure time T1, and information of all pixel reading, which are imaging conditions corresponding to the parameter “A”, are read out and temporarily stored in the register 80 as the imaging conditions of the second frame. Then, as in the first frame, the imaging control section 81 sets the gain of the amplifier 75 to G1, the exposure time of the pixel section 70 to T1, and performs imaging operation through the all pixel reading for the second frame, whereby an ordinary image signals of the second frame are outputted.

Until an instruction to switch to the narrow band mode is inputted, the imaging control section 81 sequentially reads out each parameter in the sequence pattern “AAAAAAAAA”, then stores and updates imaging conditions corresponding to each parameter for one frame in the register 80, and performs imaging operation based on the imaging conditions stored in the register 80 to sequentially outputs ordinary image signals for the third and subsequent frames.

The ordinary image signals outputted from the image sensor 39 are inputted to the processor unit 18 via the image signal wiring 42b in the insertion section 11 and universal cable 13.

Then, the ordinary image signals inputted to the processor unit 18 are temporarily stored in the image input controller and then stored in the memory 53. The ordinary image signals of each frame read out from the memory 53 are subjected to tone correction processing and sharpness correction processing in the image processing section 52 and sequentially outputted to the video output section 54.

The video output section 54 performs predetermined processing on the inputted image signals to generate display control signals and sequentially outputs the display control signals of each frame to the monitor 20. The monitor 20 displays an ordinary image based on the inputted display control signals.

Next, if an instruction to perform imaging in the narrow band mode is set and inputted by the user at the input section 55 in the middle of the imaging operation in the normal mode described above, the control section 56 outputs a control signal “1”. Here, it is assumed that the control signal “1” is outputted in the middle of the imaging operation for the third frame in the ordinary mode, as shown in FIG. 10.

When the control signal “1” is received, the imaging control section 81 selects a sequence pattern “ABABABABA” from a plurality of types of sequence patterns set in the sequence pattern setting section 78.

Then, imaging conditions corresponding to the first parameter of the sequence pattern are read out by the imaging control section 81 from the imaging condition setting section 79. That is, gain G1, exposure time T1, and information of all pixel reading, which are imaging conditions corresponding to the parameter “A” are read out and temporarily stored in the register 80 by the imaging control section 81 as the imaging conditions of the fourth frame.

Here, although the switching to the narrow band mode is made in the middle of the imaging operation for the third frame in the normal mode as described above, the imaging control section 81 does not update the imaging conditions stored in the register 80 immediately but at the start of the imaging operation for the first frame after the control signal “1” is received. That is, in the example shown in FIG. 10, the imaging conditions stored in the register 80 are updated at the start of the imaging operation for the fourth frame. In this way, by updating the imaging conditions at the timing when the frame is switched, the switching of imaging conditions in the middle of imaging operation for one frame may be prevented and inclusion of image signals obtained under different imaging conditions in the image signals of one frame may be prevented.

Then, white light is also outputted from the white light source 60 at the start of imaging the fourth frame and the image sensor 39 is controlled based on the imaging conditions in the register 80 stored by the imaging control section 81, whereby an imaging operation is performed. Note that the imaging operation at this time is identical to that of the ordinary mode described above.

Next, imaging conditions corresponding to the second parameter of the sequence pattern are read out by the imaging control section 81 from the imaging condition setting section 79. That is, gain G2, exposure time T2, and information of every other line reading, which are imaging conditions corresponding to the parameter “B” are read out and temporarily stored in the register 80 by the imaging control section 81 as the imaging conditions of the fifth frame.

Then, a switching is made to the projection of narrow band light from the special light source 61 in the fifth frame, and the gain of the amplifier 75 is set to G2, the exposure time of the pixel section 70 is set to T2, and an imaging operation is performed through every other line reading, whereby narrow band image signals are outputted, as shown in FIG. 10.

Then, imaging conditions corresponding to the third parameter of the sequence pattern are read out by the imaging control section 81 from the imaging condition setting section 79. That is, gain G1, exposure time T1, and information of all pixel reading, which are imaging conditions corresponding to the parameter “A” are read out and temporarily stored in the register 80 by the imaging control section 81 as the imaging conditions of the sixth frame.

Then, a switching is made to the projection of white light from the white light source 60 again in the sixth frame, and the imaging operation identical to that of the ordinary mode is performed again and ordinary image signals are outputted from the image sensor 39.

As described above, in the narrow band mode, the projection of white light and the projection of narrow band light are switched alternately on a frame-by-frame basis and imaging operations are performed by alternately switching the imaging conditions corresponding to the parameter “A” and the imaging conditions corresponding to the parameter “B” on a frame-by-frame basis by the imaging control section 81. This causes ordinary image signals and narrow band image signals to be outputted alternately from the image sensor 39 on a frame-by-frame basis.

The ordinary image signals and narrow band image signals alternately outputted from the image sensor 39 are inputted to the processor unit 18 via the image signal wiring 42b in the insertion section 11 and universal cable 13.

Then, the ordinary image signals and the narrow band image signals inputted to the processor unit 18 are temporarily and sequentially stored in the image input controller and then stored in the memory 53. Then, the ordinary image signals and the narrow band image signals are read out from the memory 53 on a frame-by-frame basis and subjected to tone correction processing and sharpness correction processing in the image processing section 52, and then sequentially outputted to the video output section 54.

The video output section 54 performs predetermined processing on the inputted image signals to generate display control signals respectively and sequentially outputs the display control signals of each frame to the monitor 20. The monitor 20 displays an ordinary image and a narrow band image separately based on the inputted display control signals of ordinary image signals and display control signals of narrow band image signals.

According to the endoscope system of the aforementioned embodiment, combinations of sequence pattern and control signal are preset in the sequence pattern setting section 78, a plurality of types of parameters and imaging conditions corresponding to each parameter are associated and set in the imaging condition setting section 79, and the imaging control section 81 obtains a sequence pattern based on the control signal outputted from the control section 56 of the processor unit 18, then sequentially reads out imaging conditions corresponding to each parameter included in the obtained sequence pattern from the imaging condition setting section 79, and controls the imaging operation of the image sensor 39 on a frame-by-frame basis or on a plurality of frames-by-frames basis based on the sequentially read out imaging conditions. This requires output of control signal from the processor unit 18 to the endoscope unit 10 only once for mode switching in the beginning, and eliminates the need to perform communication of control signal on a frame-by-frame basis as in the past.

Consequently, an image collapse due to a change in the imaging conditions in the middle of imaging one frame arising from a receiving error of control signal may be prevented and the burden on the control section 56 of the processor unit 18 may be reduced at the same time.

Here, in the endoscope system of the aforementioned embodiment, if, for example, the control signal “1” for switching to the narrow band mode is outputted from the control section 56 of the processor unit 18 immediately before the frame is switched, as illustrated in FIG. 12, there may be a case in which the update of the imaging conditions in the register 80 of the image sensor 39 may not be made by the imaging of the next frame. In such a case, if, for example, the sequence pattern of the narrow band mode is “BABABABA”, the imaging operation for the fourth frame should be performed under the imaging conditions corresponding to the parameter “B” but the imaging conditions of the fourth frame become those corresponding to the parameter “A” of the third frame due to the update delay. That is, the setting of the imaging conditions is shifted by one frame and imaging conditions for imaging a narrow band image are set when the white light is projected while the imaging conditions for imaging an ordinary image are set when the narrow band light is projected, whereby appropriate images are not displayed.

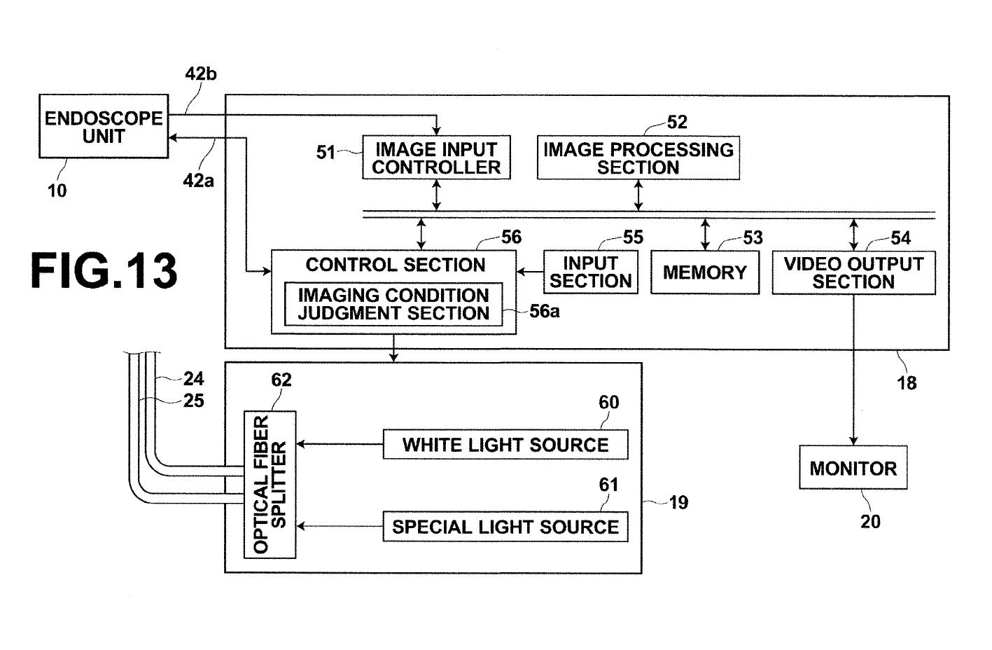

Consequently, an arrangement may be adopted in which imaging conditions currently set in the register 80 or the parameter corresponding to the imaging conditions is outputted to the control section 56 of the processor unit 18 at the start of imaging each frame and a judgment is made in an imaging condition judgment section 56a of the control section 56 shown in FIG. 13 whether or not the imaging conditions or the parameter outputted from the imaging control section 81 is correct. Then, if a judgment is made by the imaging condition judgment section 56a that the parameter is not correct and if, for example, the imaging conditions are shifted by one frame as described above, the light source unit 19 may be controlled such that the timing of the white light and narrow band light outputted from the light source unit 19 is shifted by one frame.

The judgment in the imaging condition judgment section 56a may be made, for example, by presetting imaging conditions or the parameter corresponding to each frame based on the projection timing of white light and narrow band light in the imaging condition judgment section 56a and making a comparison between the preset imaging conditions or the parameter corresponding to each frame and the imaging conditions or the parameter outputted from the imaging control section 81 to determine if they agree or not.

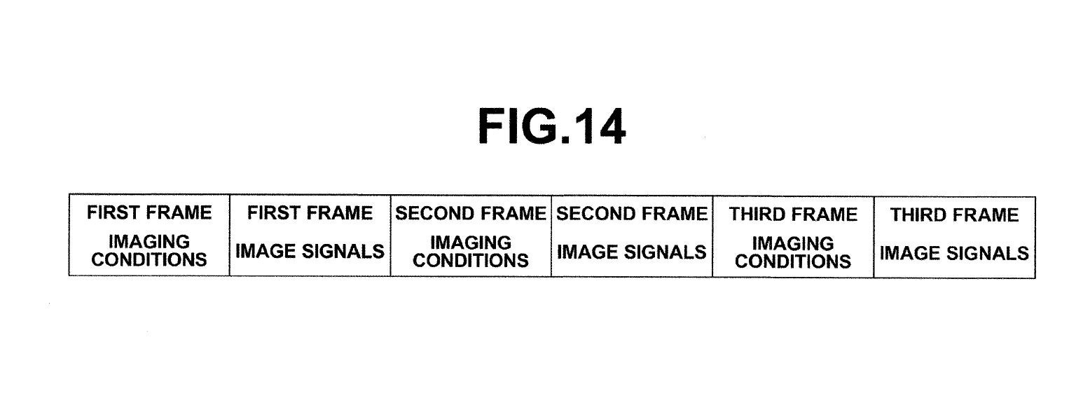

The imaging conditions or the parameter to be outputted from the imaging control section 81 may be outputted to the processor unit 18 via the control signal wiring in the multi-core cable 28 or via the image signal wiring by superimposing on the image signal. One of the methods for superimposing the imaging conditions or the parameter on the image signal, for example, is to output the imaging conditions or the parameter during the blanking time between each frame, as shown in FIG. 14.

In the endoscope system of the aforementioned embodiment, the every other line reading is implemented with an exposure time of 1/30 sec as the imaging conditions of the narrow band image, but not limited to this and, for example, the exposure time is set to 1/60 sec as in the ordinary image and each even row may be read out two times. Alternatively, a so-called binning reading may be performed in which, for example, G and B filter signals in the second row and G and B filter signals in the fourth row shown by the thick frames in FIG. 11 are read out at the same time and the signals of G filters in the second and fourth rows are added together while the signals of B filters in the second and fourth rows are added together. Note that the binning reading is not limited to the ranges illustrated by the thick frames in FIG. 11 and the identical binning reading may be performed for other ranges of the pixel section 70.

Further, in the endoscope system of the aforementioned embodiment, the narrowed blue light and green light are described as the special light emitted from the special light source 61, but not limited to these. For example, the special light source 61 may be an excitation light source that emits blue excitation light or purple excitation light having a shorter wavelength than the blue light in order to detect red fluorescence or green fluorescence from the fluorescent agent administered to an observation target area or autofluorescence in the range from green to red emitted from a living body itself. Also in the case where such fluorescence is detected, it is preferable that the gain of the amplifier 75 is increased as the intensity of the fluorescence is very weak.

Then, as the sequence pattern corresponding to the fluorescence mode, “ABABABABA” shown in FIG. 6 is set in which the parameter “A” is set as the parameter corresponding to imaging conditions when the white light is projected while the parameter “B” is set as the parameter corresponding to imaging conditions when the excitation light is projected. As for the imaging conditions corresponding to the parameter “B”, the gain G2 of the amplifier 75 is preferable to be increased to a value greater than that in the case of the narrow band mode. As for the exposure time T2 of the pixel section 70, a value twice that of the ordinary mode may be set and the reading target pixel may be the every other line reading to read only odd rows on which R (red) and G (blue) filters that transmit fluorescent light are disposed. The sequence patterns are not limited to those described above, and excitation light may be continuously projected and the sequence pattern may be set as “CCCCCCCCC” in which the aforementioned imaging conditions for imaging fluorescence images may be set as the imaging conditions corresponding to the parameter “C”.

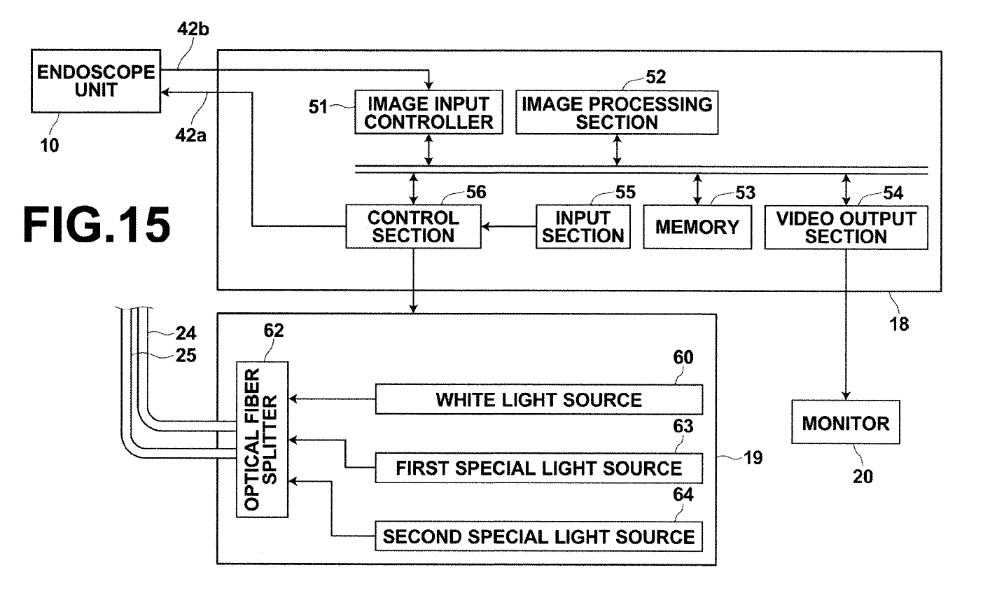

Further, as illustrated in FIG. 15, two special light sources of a first special light source 63 and a second special light source 64 may be provided, in which the first light source 63 is the narrow band light source while the second light source 64 is the excitation light source. Then, as the combination, for example, of control signal corresponding to the narrow band/fluorescence mode and sequence pattern, the combination of “2” and “ABCABCABC” shown in FIG. 6 may be set. For example, in this case, the parameter “A” may be set as the parameter corresponding to the imaging conditions when the white light is projected, the parameter “B” may be set as the parameter corresponding to the imaging conditions when the narrow band light is projected, and the parameter “C” may be set as the parameter corresponding to the imaging conditions when the excitation light is projected.

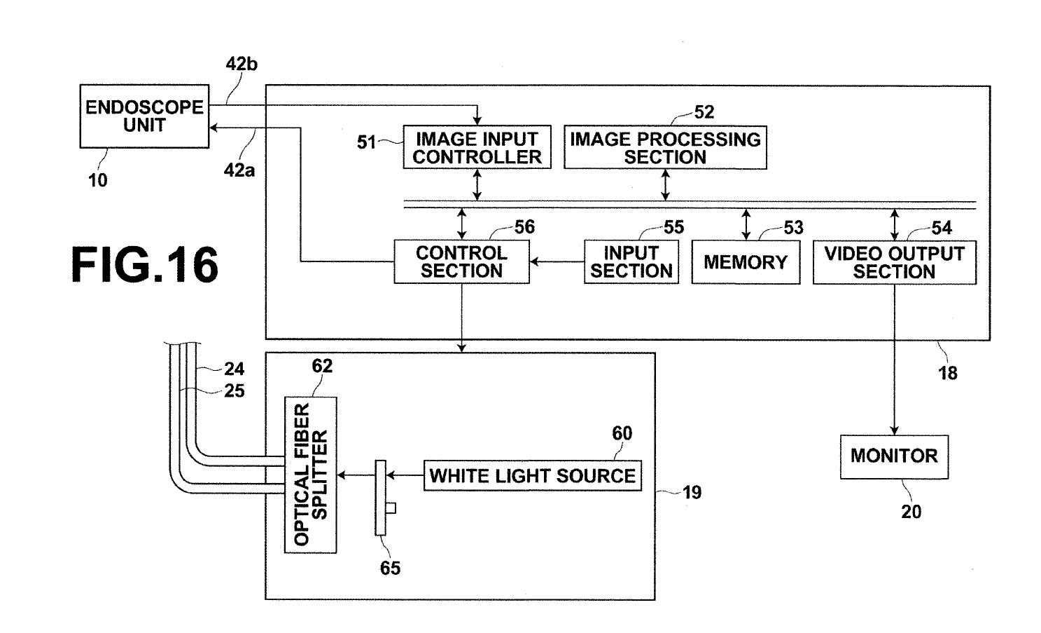

Further, in the endoscope system of the aforementioned embodiment, the white light source and the special light source are provided as a plurality of types of light sources, but the white light source 60 and an RGB frame sequential filter 65 may be provided, as illustrated in FIG. 16, to provide, in effect, three light sources of a red light source, a green light source, and a blue light source.

Then, as the combination of control signal and sequence pattern, for example, the combination of “2” and “ABCABCABC” shown in FIG. 6 may be set. In this case, for example, the parameter “A” may be set as the parameter corresponding to the imaging conditions when the red light is projected, the parameter “B” may be set as the parameter corresponding to the imaging conditions when the green light is projected, and the parameter “C” may be set as the parameter corresponding to the imaging conditions when the blue light is projected.

As for the imaging conditions when the red light, the green light, and the blue light are projected, for example, gains G1, G2, G3 and exposure times T1, T2, T3 which are different from each other may be set. Further, by performing binning reading by changing the reading target pixel depending on the color and pixel addition may or may not be performed depending on the color.

In the case where the output of a particular color light source of the red, green, and blue light sources is weak, gain increase in the image sensor, extension of the exposure time, or performance of pixel addition when the weak color light is projected may result in an image of a higher S/N ratio than in the case in which the gain of the color is increased in the processor 18 in the latter stage.

Further, the performance or non-performance of binning reading is included as one of the imaging conditions in the aforementioned embodiment but not limited to this and, for example, whether or not a plurality of pixel signals is outputted by averaging them may be set as one of the imaging conditions. More specifically, whether or not the average value of two pixel signals is outputted is set as one of the imaging conditions.

The light source types, imaging conditions corresponding to each of the light sources, and sequence patterns are not limited to the examples described in the aforementioned embodiments and may be changed depending on the application.

What is claimed is:

1. An endoscope system, comprising a light source unit that emits light by sequentially switching a plurality of types of light sources, an endoscope unit equipped with an image sensor, and a processor unit connected to the endoscope unit as an external unit and includes a control section that controls the endoscope unit, wherein the endoscope unit comprises:
a sequence pattern setting section in which a combination of sequence pattern and predetermined control signal to be outputted from the control section is set, the sequence pattern including a plurality of parameters, each corresponding to imaging conditions of the image sensor on a frame-by-frame or a plurality of frames-by-frames basis according to the type of light source;
an imaging condition setting section in which a plurality of types of parameters and imaging conditions corresponding to each parameter are set in association with each other; and
an imaging control section that obtains the sequence pattern based on the control signal outputted from the control section of the processor unit, sequentially reads out the imaging conditions corresponding to each parameter included in the obtained sequence pattern from the imaging condition setting section, and controls the imaging operation of the image sensor on a frame-by-frame or a plurality of frames-by-frames basis based on the sequentially read out imaging conditions.

2. The endoscope system as claimed in claim 1, wherein:
the sequence pattern setting section is a section in which a plurality of types of combinations of sequence pattern and control signal is set; and
the imaging control section selects and obtains one sequence pattern from the plurality of types of sequence patterns.

3. The endoscope system as claimed in claim 1, wherein the endoscope unit comprises a register in which the imaging conditions sequentially read out from the imaging condition setting section on a frame-by-frame or a plurality of frames-by-frames basis are temporarily stored and sequentially updated.

4. The endoscope system as claimed in claim 1, wherein the imaging control section outputs information of the currently set imaging conditions to the processor unit.

5. The endoscope system as claimed in claim 4, wherein the imaging control section outputs the information of the imaging conditions by superimposing the information on an image signal outputted from the image sensor.

6. The endoscope system as claimed in claim 5, wherein the imaging control section outputs the information of the imaging conditions during a blanking time of the image sensor.

7. The endoscope system as claimed in claim 4, wherein the processor unit comprises an imaging condition judgment section that judges whether or not the information of the imaging conditions outputted from the imaging control section is correct.

8. The endoscope system as claimed in claim 1, wherein:
the endoscope unit comprises an amplifier that amplifies an image signal outputted from the image sensor; and
one of the imaging conditions is gain of the amplifier.

9. The endoscope system as claimed in claim 1, wherein the imaging conditions include at least one of exposure time of the image sensor and reading target pixel information of the image sensor.

10. The endoscope system as claimed in claim 9, wherein the reading target pixel information is information of reading a plurality of pixel signals by adding the signals together or information of reading a plurality of pixel signals by averaging the signals.

11. The endoscope system as claimed in claim 1, wherein at least one of the sequence pattern setting section, the imaging condition setting section, and the imaging control section is formed on one IC (Integrated Circuit) chip with the image sensor.

12. The endoscope system as claimed in claim 1, wherein the image sensor is a CMOS (Complementary Metal-Oxide Semiconductor).

13. The endoscope system as claimed in claim 1, wherein the light source unit comprises at least two of a white light source, a narrow band light source that emits narrow band light, and an excitation light source that emits excitation light for causing an observation target area to generate fluorescence.

14. The endoscope system as claimed in claim 1, wherein the light source unit comprises a red light source that emits red light, a green light source that emits green light, and a blue light source that emits blue light, as the plurality of types of light sources.
